(19) United States
(12) Patent Application Publication
(10) Pub. No.: US 2013/0165786 A1
(43) Pub. Date: Jun. 27, 2013

(54) SYSTEMS AND METHODS FOR OPTICAL POWER AND DATA TRANSMISSION IN ULTRASOUND IMAGING

(71) Applicants: Bruno Hans Haider, Ballston Lake, NY (US); David William Vernooy, Niskayuna, NY (US)

(72) Inventors: Bruno Hans Haider, Ballston Lake, NY (US); David William Vernooy, Niskayuna, NY (US)

(73) Assignee: General Electronic Company, Schenectady, NY (US)

(21) Appl. No.: 13/333,755

(22) Filed: Dec. 21, 2011

(51) Int. Cl. A61B 8/14; A61B 8/00

(52) U.S. Cl. 600/443; 600/459

(57) ABSTRACT

An ultrasound system includes an optical conduit adapted to transmit an optical signal between a first end of the optical conduit and a second end of the optical conduit. The ultrasound system also includes a console coupled to the first end of the optical conduit and having an optical power source adapted to generate the optical signal. Further, the ultrasound system includes an ultrasound probe coupled to the second end of the optical conduit and having power conversion circuitry adapted to receive the optical signal and to convert the optical signal into electrical power.

### BACKGROUND

The subject matter disclosed herein relates generally to ultrasound imaging, and, more particularly, to optical data and power transmission between components of ultrasound imaging systems.

Medical diagnostic ultrasound is an imaging modality that employs ultrasound waves to probe the acoustic properties of the body of a patient and to produce a corresponding image. Generation of sound wave pulses and detection of returning echoes is typically accomplished via a plurality of transducers located in the probe. Such transducers typically include electromechanical elements capable of converting electrical energy into mechanical energy for transmission and mechanical energy back into electrical energy for receiving purposes. Some ultrasound probes include up to thousands of transducers arranged as linear arrays or a 2D matrix of elements.

In addition to the ultrasound probe, ultrasound imaging systems typically also include a console having electrical circuitry that is capable of processing the electrical signals detected by the transducers and, if desired, displaying an image corresponding to the patient's anatomy. In certain systems, the console may also provide the ultrasound probe the energy necessary to power the electronics in the probe. Accordingly, ultrasound imaging systems typically include a cable that communicatively couples the probe to the console, thus enabling the transmission of data and power between the system components. Unfortunately, this cable is often bulky and adds to the overall size and weight of the ultrasound system.

### BRIEF DESCRIPTION

In one embodiment, an ultrasound system includes an optical conduit adapted to transmit an optical signal between a first end of the optical conduit and a second end of the optical conduit. The ultrasound system also includes a console coupled to the first end of the optical conduit and having an optical power source adapted to generate the optical signal. The ultrasound system further includes an ultrasound probe coupled to the second end of the optical conduit and having power conversion circuitry adapted to receive the optical signal and to convert the optical signal into electrical power.

In another embodiment, an ultrasound system includes an optical conduit adapted to transmit a modulated optical signal between a first end of the optical conduit and a second end of the optical conduit. The ultrasound system also includes an ultrasound probe coupled to a first end of the optical conduit and having a plurality of transducer elements adapted to sense an ultrasound signal and to convert the ultrasound signal into a first electrical signal. The ultrasound system also includes receiving circuitry adapted to receive the first electrical signal and to process the first electrical signal to produce a processed electrical signal. The ultrasound system also includes an optical generation and modulation device disposed within the ultrasound probe and adapted to receive the processed electrical signal and to produce the modulated optical signal. The ultrasound system also includes a console coupled to the second end of the optical conduit and having detection circuitry adapted to receive the modulated optical signal and to convert the modulated optical signal into a second electrical signal corresponding to the processed electrical signal.

In another embodiment, an ultrasound system includes an ultrasound probe that includes an array of transducers adapted to sense an ultrasound signal and to convert the ultrasound signal into an electrical signal. The ultrasound probe also includes an optical generation and modulation device adapted to receive the electrical signal, and to produce a modulated optical signal.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic diagram illustrating an embodiment of an ultrasound system capable of optically transmitting power and data between an ultrasound probe and a console;

FIG. 2 is a schematic diagram illustrating an embodiment of an ultrasound system capable of converting optical power into electrical power within a connector that couples an optical conduit to a console;

FIG. 3 illustrates an embodiment of a method that may be implemented by a controller to optically transmit power from an ultrasound console to an ultrasound probe;

FIG. 4 illustrates an embodiment of a method that may be implemented by a controller to optically transfer ultrasound signals from an ultrasound probe to a console;

FIG. 5 is a schematic illustrating optical data transmission of multiplexed electrical signals from an ultrasound probe to an ultrasound console in accordance with an embodiment of presently disclosed techniques; and

FIG. 6 is a schematic illustrating optical data transmission of modulated and multiplexed electrical signals from an ultrasound probe to an ultrasound console in accordance with an embodiment of presently disclosed techniques.

### DETAILED DESCRIPTION

As described in detail below, provided herein are embodiments of ultrasound systems including an ultrasound probe and a console capable of optically communicating and exchanging power with one another via an optical conduit. For example, in some embodiments, energy for powering the electronics located in the probe may be transferred from an optical power source in the console to power conversion circuitry in the probe via the optical conduit. In such embodiments, the optical energy may be converted into electrical energy by the power conversion circuitry in the probe. Still further, in certain embodiments, transfer of ultrasound signals from the probe to the console may be accomplished via inclusion of an optical generation and modulation device in the probe. That is, in these embodiments, the ultrasound signals may be converted to an electrical signal when received by a transducer, and the electrical signal may be processed and/or combined with additional electrical signals before a modulated optical signal is produced by the optical generation and modulation device and transferred to the console via the optical conduit. As such, presently disclosed embodiments may provide for the optical transmission of both data and power between the probe and the console during operation of the ultrasound system. The foregoing feature may offer advantages over traditional systems by reducing or eliminating the need for oversized cables coupling the probe to the console, thereby enabling connection between the probe and the console to be established with a reduced cable size and weight.

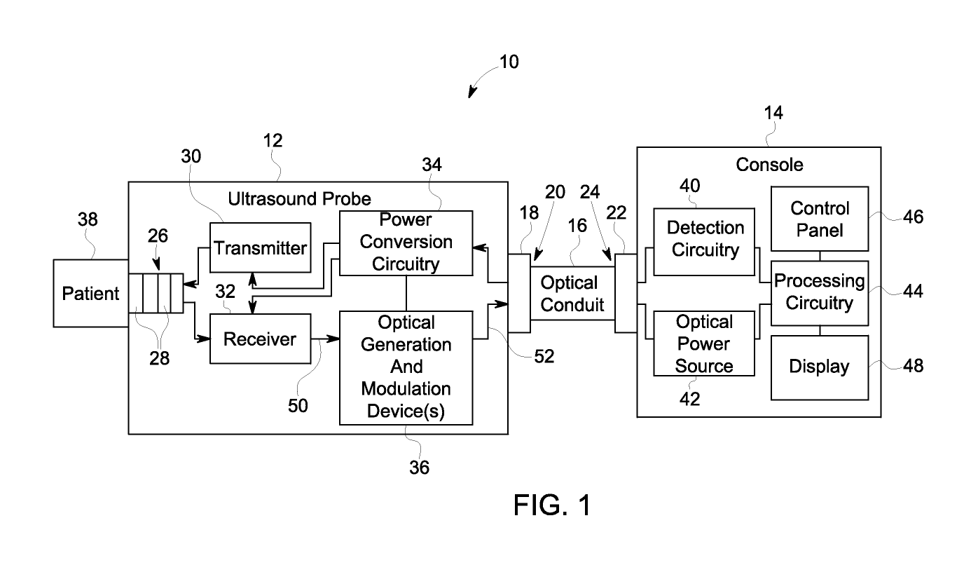

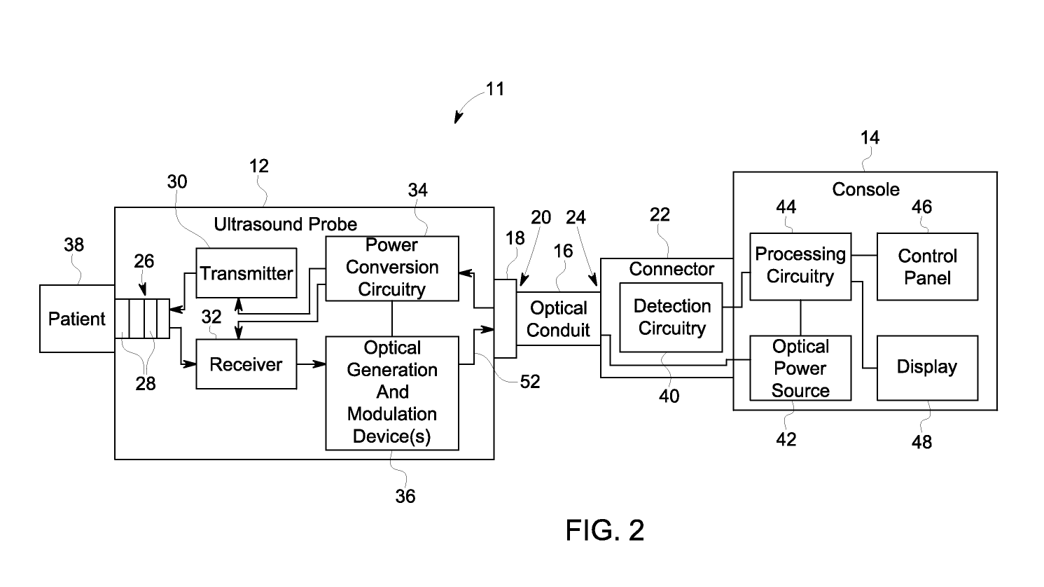

Turning now to the drawings, FIGS. 1 and 2 are block diagrams of embodiments of ultrasound systems 10 and 11 including an ultrasound probe 12 (hereinafter, “the probe”) and a console 14 coupled together by an optical conduit 16. Specifically, in the illustrated embodiment, a probe connector 18 couples the probe 12 to the optical conduit 16 at a first end 20 of the optical conduit 16. Similarly, a console connector 22 couples the console 14 to the optical conduit 16 at a second end 24 of the optical conduit 16. It should be noted that the optical conduit 16 may be any conduit suitable for transmission of optical signals or any combination of conduits capable of transmitting optical signals. For example, the optical conduit 16 may include one or more optical fibers.

In the illustrated embodiment, the probe 12 includes a transducer array 26 having a plurality of transducer elements 28, a transmitter 30, a receiver 32, power conversion circuitry 34, and an optical generation and modulation device 36. The transducer array 26 of the probe 12 is positioned on a patient 38 to probe the patient's anatomy with ultrasound signals. In some embodiments, the probe 12 may include a handle portion (e.g., a grooved section designed for gripping) that facilitates use by an operator, such as a medical technician. Additionally, it should be noted that the probe 12 may be manufactured to take on any of a number of geometries, such as a t-shape, a rectangle, a cylinder, and so forth. Further, the probe 12 is coupled to the console 14 that includes detection circuitry 40, an optical power source 42 (e.g., a laser), processing circuitry 44, a control panel 46, and a display 48. In certain embodiments, the console 14 may include additional elements not shown in FIG. 1, such as keyboards, additional data acquisition and processing controls, additional image display panels, multiple user interfaces, and so forth.

During operation, the optical conduit 16 may facilitate the bidirectional exchange of power and/or data between the probe 12 and the console 14. For instance, in some embodiments, the console 14 transmits control signals to the probe 12. In such embodiments, the detection circuitry 40 converts the electrical control signals generated by the processing circuitry 44 into optical signals before transmitting the optical signals to the probe 12 via the optical conduit 16. For further example, in certain embodiments, the processing circuitry 44 in the console 14 receives matrices of digital data representing reflection signals returned from tissue interfaces within the patient 38 during a pulse-echo data acquisition method. These matrices of digital data, or processed versions of these matrices, are transmitted to the processing circuitry 44 from the probe 12 via the optical conduit 16. As such, the electrical signal encoding the matrices of digital data, or processed matrices of digital data, is utilized to produce an optically modulated signal generated by the optical generation and modulation device 36 before being transmitted over the optical conduit 16. Once received by the console 14, the optical signals are converted into an electrical signal corresponding to the matrices of digital data or the processed matrices of digital data and transferred to the processing circuitry 44.

During an ultrasound acquisition process, the transducer array 26 of the probe 12 is positioned on the patient 38. The transmitter 30 transmits ultrasound energy into the patient 38 via the transducer elements 28 of the transducer array 26, and the receiver 32 receives data from the array of transducers 26 corresponding to matrices of data representing reflection signals returned from tissue interfaces within the patient 38 during data acquisition. The illustrated probe 12 includes the transducer array 26 of transducers 28 that are configured to produce and detect ultrasound waves. Each individual transducer 28 is generally capable of converting electrical energy into mechanical energy for transmission and mechanical energy into electrical energy for receiving purposes. In certain embodiments, the transducers 28 may be voltage biased when receiving echoes back from the patient 38. That is, the transducers 28 may be precharged to a certain voltage (e.g., 1v, 2v) prior to receiving signals back from the patient 38 such that all received signals take on a positive value. The foregoing feature may have the effect of simplifying electrical circuitry associated with the receiving cycle in certain embodiments. In some embodiments, each transducer 28 may include a piezoelectric ceramic, a matching layer, an acoustic absorber, and so forth. Additionally, the transducers 28 may be of any type suitable for use with diagnostic ultrasound, such as broad-bandwidth transducers, resonance transducers, and so forth. In the illustrated embodiment, the transducer array 26 is depicted as a 4×1 matrix of transducers 28. However, it should be noted that in other embodiments, more or fewer transducers 28 may be included in each array 26, and the transducer array 26 may include multiple sub-arrays of transducer 28 if desired for the given application.

Once the receiver 32 receives data from the array of transducers 26 corresponding to matrices of data representing reflection signals returned from tissue interfaces within the patient 38 during data acquisition, these matrices of data may be processed and communicated to the optical generation and modulation device 36 via a processed electrical signal represented by arrow 50. In some embodiments, the processed electrical signal 50 may correspond directly to the matrices of data. However, in other embodiments, the processed electrical signal 50 may correspond to compiled data received from more than one transducer element and/or data that has been processed, for example, to reduce or eliminate signal noise. The optical generation and modulation device 36 receives the processed electrical signal 50 and produces a modulated optical signal, as represented by arrow 52. That is, a single device located in the probe 12 may be utilized to produce an optically modulated signal corresponding to the data being transferred to the console 14. The foregoing feature may offer advantages over existing systems by reducing or eliminating the need for the optical power source 42 to produce the optical signal necessary for such transmission of data, thus reducing the complexity of the optical path needed to communicate the acquired data to the processing circuitry 44. The modulated optical signal 52 produced in the probe 12 is transferred to the console 14 via optical conduit 16. In certain embodiments, the optical generation and modulation device 36 may, for example, be a vertical cavity surface emitting laser (VCSEL) capable of producing the modulated optical signal 52.

Once received by the console 14, the modulated optical signal 52 is converted by the detection circuitry 40 into an electrical signal and transferred to the processing circuitry 44 for processing. Accordingly, the processing circuitry 44 may include memory, which may be volatile or non-volatile memory, such as read only memory (ROM), random access memory (RAM), magnetic storage memory, optical storage memory, and so forth, for storing and/or processing the signals. Once processed, the matrices of data may be utilized to produce an image of the patient's anatomy that is displayed on the display 48 in accordance with operator selections input via the control panel 46.

In some embodiments, the electronic components of the probe 12 are powered by the optical power source 42 located in the console 14. In these embodiments, the optical power source 42 generates an optical power signal and transmits the optical power signal to the probe 12 via the optical conduit 16. The power conversion circuitry 34, which may include an optical detector, converts the optical power signal into electrical power that powers, for example, the transmitter 30, the receiver 32, and/or the optical generation and modulation device 36. The electrical power may, for example, be used to provide an electrical voltage suitable for excitation of the transducer elements 28.

In the embodiment shown in FIG. 1, the detection circuitry 40 is located within the console 14. However, in the embodiment of FIG. 2, the detection circuitry 40 is located within the connector 22 that couples the end 24 of the optical conduit 16 to the console 14. That is, while in some embodiments the detection circuitry 40 and its associated optical detector(s) may be integral with the console 14, in other embodiments, such circuitry may be integrated with other system components and subsequently communicatively coupled to the appropriate circuitry (e.g., processing circuitry 44) located within the console 14. Further, it should be noted that in some embodiments, the power conversion circuitry 34 may be similarly located in the connector 18 instead of integrated within the probe 12. Indeed, the power conversion circuitry 34 and/or the detection circuitry 40 may be located in any suitable location within the ultrasound systems 10 and 11.

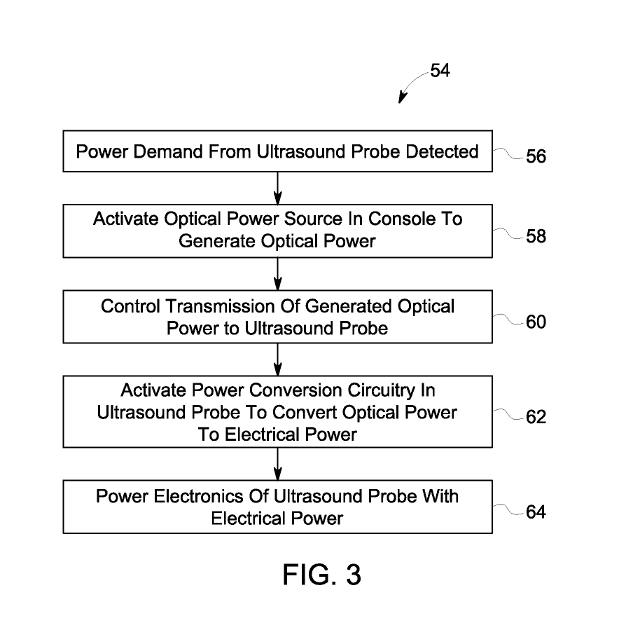

FIG. 3 illustrates a method 54 that may be implemented by a controller to optically transmit power from the ultrasound console 14 to the ultrasound probe 12 in accordance with an embodiment of presently disclosed techniques. As illustrated, the method 54 begins when a power demand originating from the ultrasound probe 12 is detected (block 56). For example, in one instance, the power demand may be a voltage demand from the transducer elements 28. Once the power demand is detected, the optical power source 42 located in the console 14 is activated to produce optical power (block 58), and the optical power is transmitted, for example, via optical conduit 16, to the ultrasound probe 12 (block 60). The power conversion circuitry 34 located in the probe 12 is then activated to convert the received optical power into electrical power (block 62). It should be noted that in some embodiments, the power conversion circuitry 34 may also include an optical detector capable of detecting the incoming optical signals from the optical conduit 16. The electrical power output from the power conversion circuitry 34 may then be utilized to power the electronic components of the probe 12 (block 64). For example, the electrical power may be used to provide an electrical voltage suitable for excitation of the transducer elements 28 in the transducer array 26.

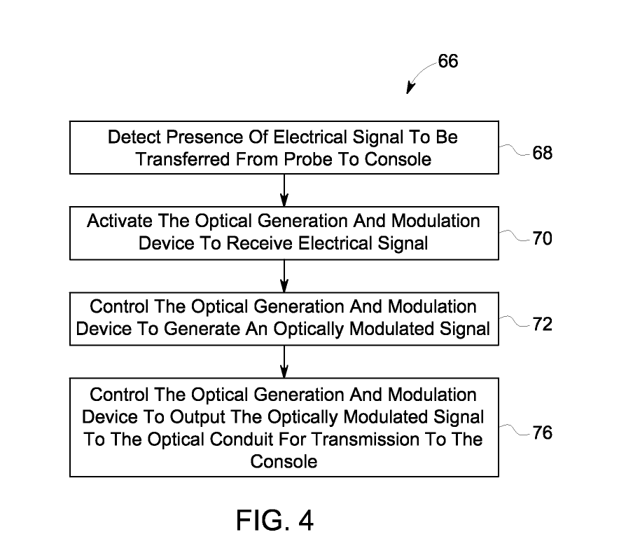

FIG. 4 illustrates a method 66 that may be implemented by a controller to optically transfer ultrasound signals from the ultrasound probe 12 to the ultrasound console 14 in accordance with an embodiment of presently disclosed techniques. The method 66 is initiated when the presence of an electrical signal to be transferred from the probe 12 to the console 14 is detected (block 68). The method 66 proceeds with activation of the optical generation and modulation device 36 for receiving the electrical signal (block 70). Once the electrical signal is received, the optical generation and modulation device 36 is controlled to produce a suitable optically modulated signal (block 72). The optically modulated signal may encode, for example, matrices of data representing reflection signals returned from tissue interfaces within the patient 38 after probing with ultrasonic energy and being processed by receiving circuitry. The optical generation and modulation device 36 is then controlled to output the optically modulated signal to the optical conduit 16 for transmission to the console 14 (block 76).

The foregoing feature of presently disclosed embodiments may offer advantages over approaches utilizing an optical power generation source in the console and a modulator in the probe. For example, such an approach where power generation and signal modulation are split may utilize transmission of a laser output from the console to the probe, modulation of the laser signal in the probe, and subsequent transmission of the modulated signal back to the console in order to transfer data optically from the probe to the console. However, in presently disclosed embodiments, optical transmission of data may be simplified since the optical generation and modulation device 36 is capable of both producing an optical output as well as modulating the signal prior to transmission across the optical conduit 16.

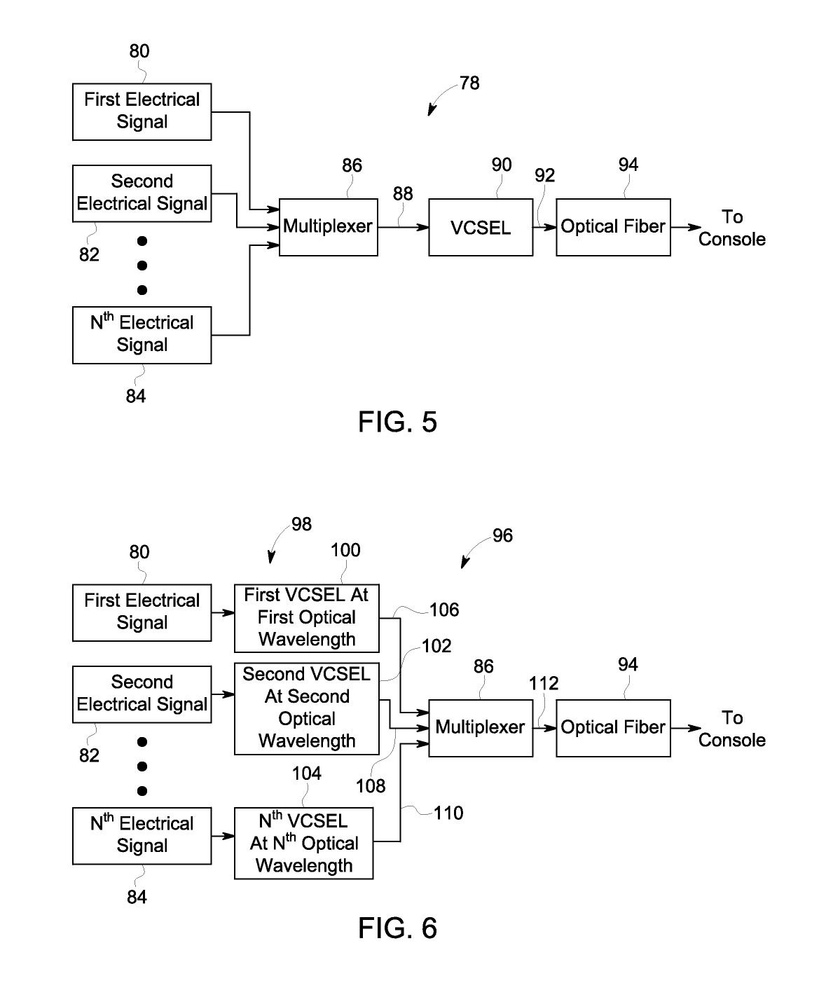

In certain embodiments, it may be desirable to transfer multiple signals over the same optical fiber, and such a feature may be enabled by inclusion of one or more multiplexers into the ultrasound systems 10 and 11. For example, FIG. 5 is a schematic 78 illustrating optical data transmission of multiplexed signals from the ultrasound probe 12 to the ultrasound console 14 in accordance with an embodiment of the presently disclosed techniques. As shown, electrical signals 80, 82, and 84 are directed into a multiplexer 86 that produces a single output represented by arrow 88. The multiplexed output 88 is directed to the optical generation and modulation device 36, which is a VCSEL 90 in the illustrated embodiment. As before, the VCSEL generates an optically modulated signal corresponding to the multiplexed output 88 to produce a modulated optical output represented by arrow 92. This output 92 is then transferred to an optical fiber 94 for transmission to the console 14. In this way, multiple signals may be transferred from the probe 12 to the console 14 via a single optical fiber 94.

For further example, in the alternate embodiment illustrated in a schematic 96 of FIG. 6, a plurality 98 of optical generation and modulation devices, which are VCSELs in the illustrated embodiment, are provided. In this embodiment, the first electrical signal 80 is received by a first VCSEL 100 operating at a first optical wavelength, the second electrical signal 82 is received by a second VCSEL 102 operating at a second optical wavelength, and the n^{th} electrical signal 84 is received by an n^{th} VCSEL 104 operating at an n^{th} optical wavelength. The optically modulated outputs 106, 108, and 110 of the VCSELs 100, 102, and 104, respectively, are transferred to the multiplexer 86. The multiplexer 86 generates a single output 112 that is communicated to the console 14 via the optical fiber 94. Here again, multiple electrical signals may be transferred from the probe 12 to the console 14 via one optical fiber 94, thus reducing the bulk necessary in the cable or conduit 16 that couples the probe 12 to the console 14.

This written description uses examples to disclose the relevant subject matter, including the best mode, and also to enable any person skilled in the art to practice the present approach, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound system, comprising:
an optical conduit configured to transmit an optical signal between a first end of the optical conduit and a second end of the optical conduit;
a console coupled to the first end of the optical conduit and comprising an optical power source configured to generate the optical signal; and
an ultrasound probe coupled to the second end of the optical conduit and comprising power conversion circuitry configured to receive the optical signal and to convert the optical signal into electrical power.

2. The ultrasound system of claim 1, wherein the ultrasound probe comprises a transmitter coupled to a plurality of transducers configured to transmit an ultrasonic signal into a subject.

3. The ultrasound system of claim 2, wherein the transmitter is configured to receive the electrical power from the power conversion circuitry and to provide at least a portion of the electrical power to at least one of the plurality of transducers.

4. The ultrasound system of claim 2, wherein at least one of the plurality of transducers is configured to receive a signal corresponding to the ultrasonic signal after interaction with the subject, and wherein the ultrasound probe further comprises a receiver configured to receive the signal from at least one of the plurality of transducers.

5. The ultrasound system of claim 1, wherein the ultrasound probe comprises an optical generation and modulation device configured to receive an electrical signal corresponding to an ultrasonic signal produced by a transducer array after interaction with a subject, and to produce a modulated optical signal.

6. The ultrasound system of claim 5, wherein the optical conduit is configured to transmit the modulated optical signal from the probe to the console.

7. The ultrasound system of claim 6, wherein the console comprises detection circuitry configured to receive the modulated optical signal and to convert the modulated optical signal into an electrical signal corresponding to the ultrasonic signal produced by the transducer array after interaction with the subject.

8. The ultrasound system of claim 5, wherein the optical generation and modulation device comprises a vertical cavity surface emitting laser.

9. An ultrasound system, comprising:
an optical conduit configured to transmit a modulated optical signal between a first end of the optical conduit and a second end of the optical conduit;
an ultrasound probe coupled to a first end of the optical conduit and comprising a plurality of transducer elements configured to sense an ultrasound signal and to convert the ultrasound signal into a first electrical signal;
receiving circuitry configured to receive the first electrical signal and to process the first electrical signal to produce a processed electrical signal;
an optical generation and modulation device disposed within the ultrasound probe and configured to receive the processed electrical signal and to produce the modulated optical signal; and
a console coupled to the second end of the optical conduit and comprising detection circuitry configured to receive the modulated optical signal and to convert the modulated optical signal into a second electrical signal corresponding to the processed electrical signal.

10. The ultrasound system of claim 9, wherein the console comprises an optical power source configured to generate optical power and to transmit the optical power to the ultrasound probe via the optical conduit, wherein the ultrasound probe comprises power conversion circuitry configured to receive the optical power from the optical conduit and to convert the optical power to electrical power, and wherein the ultrasound probe is configured to utilize the electrical power to transmit an ultrasonic signal into a subject.

11. The ultrasound system of claim 9, wherein the processed electrical signal comprises an electrical signal corresponding to a plurality of received and processed electrical signals from the plurality of transducer elements.

12. The ultrasound system of claim 11, wherein the ultrasound probe is configured to utilize the electrical power to provide an electrical voltage suitable for excitation of at least one of the plurality of transducer elements.

13. The ultrasound system of claim 9, wherein the ultrasound signal corresponds to an image of a patient's anatomy, and the console comprises processing circuitry configured to receive the second electrical signal from the detection circuitry and to process the second electrical signal to generate the image of the patient's anatomy.

14. The ultrasound system of claim 13, comprising a display configured to display the image of the patient's anatomy.

15. The ultrasound system of claim 9, wherein the console comprises a control panel configured to receive one or more inputs from an operator corresponding to operational parameters of the ultrasound probe.

16. The ultrasound system of claim 9, wherein the optical generation and modulation device comprises a vertical cavity surface emitting laser.

17. An ultrasound system, comprising:
an ultrasound probe, comprising:
an array of transducers configured to sense an ultrasound signal and to convert the ultrasound signal into an electrical signal; and
an optical generation and modulation device configured to receive the electrical signal, and to produce a modulated optical signal corresponding to the ultrasound signal.

18. The ultrasound system of claim 17, wherein the ultrasound probe comprises a receiver configured to receive the electrical signal from the array of transducers, to process the electrical signal to produce a processed electrical signal, and to transmit the processed electrical signal to the optical generation and modulation device.

19. The ultrasound system of claim 17, comprising an optical conduit and a console, wherein the optical conduit is configured to transmit the modulated optical signal to detection circuitry disposed within the console.

20. The ultrasound system of claim 17, wherein the optical generation and modulation device comprises a vertical cavity surface emitting laser.
